(19) United States
(12) Patent Application Publication
(10) Pub. No.: US 2023/0225895 A1
(43) Pub. Date: Jul. 20, 2023

(54) OSTOMY POUCH EMPTYING DEVICE AND METHOD OF USE

(71) Applicant: Catenation Enterprises, LLC, Lee's Summit, MO (US)

(72) Inventors: Cathy Brown Barr, Lee's Summit, MO (US); Bruce Barr, Lee's Summit, MO (US); Dan Ball, Harrisonville, MO (US)

(21) Appl. No.: 18/097,361

(22) Filed: Jan. 16, 2023

Related U.S. Application Data
(60) Provisional application No. 63/299,780, filed on Jan. 14, 2022.
(60) Provisional application No. 63/396,737, filed on Aug. 10, 2022.

(51) Int. Cl. A61F 5/44; A61F 5/445

(52) U.S. Cl. CPC A61F 5/4404; A61F 5/445

(57) ABSTRACT

An ostomy pouch emptying device which has a receiver for receiving effluent from an ostomy pouch. The emptying device may accommodate disposable receptacles, such as emesis bags with integrated rims, or may directly interface with a receptacle such as a toilet bowl. The device can be used while standing or sitting, and may be secured to a wall or placed atop a lap of the user. An embodiment could also provide a portable system for disposing of the effluent from the ostomy pouch.

### CROSS-REFERENCE TO RELATED APPLICATION

This application claims priority in U.S. Provisional Patent Application No. 63/299,780 Filed Jan. 14, 2022, and claims priority in U.S. Provisional Patent Application No. 63/396,737 Filed Aug. 10, 2022, both of which is incorporated herein by reference.

### BACKGROUND OF THE INVENTION

### 1. Field of the Invention

The present invention relates generally to an ostomy pouch emptying device and method for use thereof, and more specifically to an ostomy pouch emptying device which provides the user the ability to be self-reliant while simplifying the emptying process.

### 2. Description of the Related Art

Emptying the waste contents/effluent from an ostomy pouch can be an unpleasant and sometimes difficult, task for the ostomate and for caregivers who may be nurses or spouses or other family or friends. What is needed is a solution for providing the ostomate a simple means for independently emptying the effluent from the ostomy pouch, and for alleviating issues regarding cleaning effluent from the disposal receptacle.

Heretofore there has not been available a system or method for an ostomy pouch emptying device with the advantages and features of the present invention.

### BRIEF SUMMARY OF THE INVENTION

The present invention generally provides an ostomy pouch emptying device which has a top chamber for receiving the contents of the ostomy pouch, a central tube, and a bottom chamber which can be placed in a toilet or other receptacle for disposing of the contents of the ostomy pouch.

In one embodiment, the top end may be kidney shaped to help facilitate the placement of the top end about the body of the ostomate for easier emptying of the ostomy pouch.

In one embodiment, the tube may be flexible, allowing it to collapse and be used for a transportable, portable option to facilitate the process of emptying the ostomy pouch when the ostomate is in various settings, whether at home, at work, in public restrooms, or traveling.

In one embodiment the system may incorporate a board which can be secured to a wall or placed atop a user's lap during use.

### BRIEF DESCRIPTION OF THE DRAWINGS

The drawings constitute a part of this specification and include exemplary embodiments of the present invention illustrating various objects and features thereof.

FIG. 1 is a three-dimensional isometric view of a first embodiment of the present invention.

FIG. 2 is a partially exploded three-dimensional isometric view thereof.

FIG. 3 is a three-dimensional isometric view of the embodiment thereof shown in a typical environment of disposing of effluent from an ostomy pouch into a toilet or other disposal receptacle.

FIG. 4 is a three-dimensional isometric view of a second embodiment of the present invention shown being installed in a typical environment of a wall-mounted track, utilizing a disposable emesis bag with an integrated rim as the disposal receptacle.

FIG. 5 is three-dimensional isometric view thereof shown in an installed orientation.

FIG. 6 is a detailed view thereof taken about the circle of FIG. 5.

FIG. 7 is a three-dimensional isometric view of a third slightly alternative embodiment thereof

FIG. 8 is a three-dimensional isometric view of a fourth embodiment of the present invention.

FIG. 9 is a top plan view thereof shown in use with an ostomy pouch.

FIG. 10 is a front elevational view thereof showing the installation of a disposable emesis bag with an integrated rim within a receiver opening.

FIG. 11 is a front elevational view thereof shown with the disposable emesis bag with integrated rim installed.

FIG. 12 is a three-dimensional partially exploded isometric view of a fifth embodiment of the present invention.

FIG. 13 is a three-dimensional isometric view thereof shown assembled and partially cut-away to show interior elements thereof, including a disposable emesis bag with integrated rim.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. First Embodiment Ostomy Disposal System 2

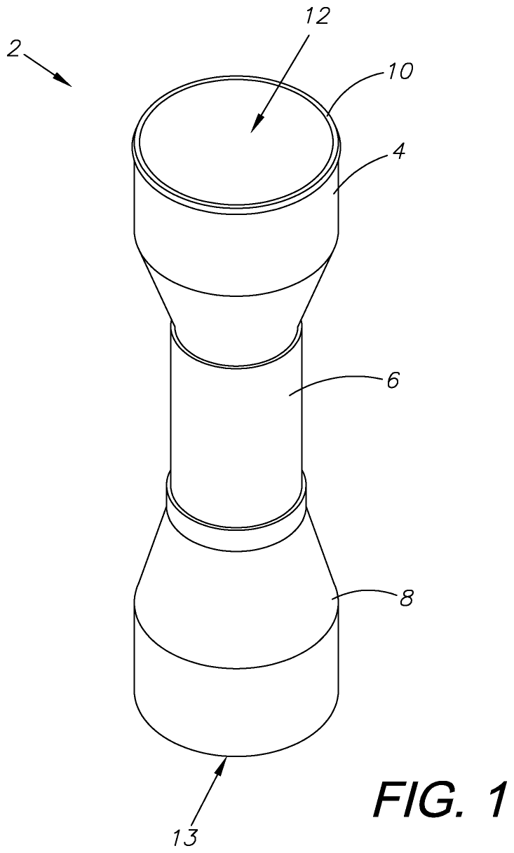

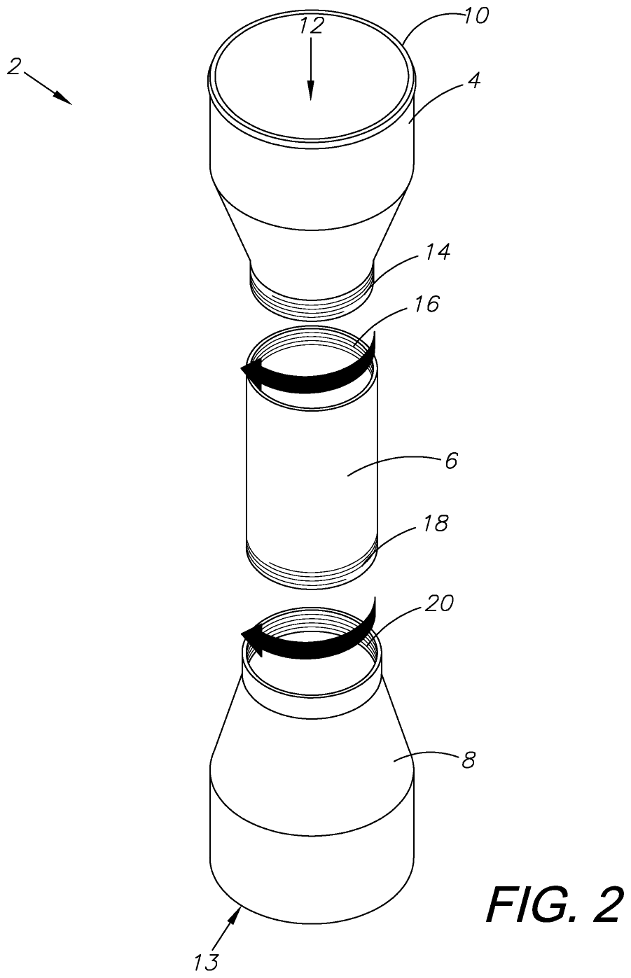

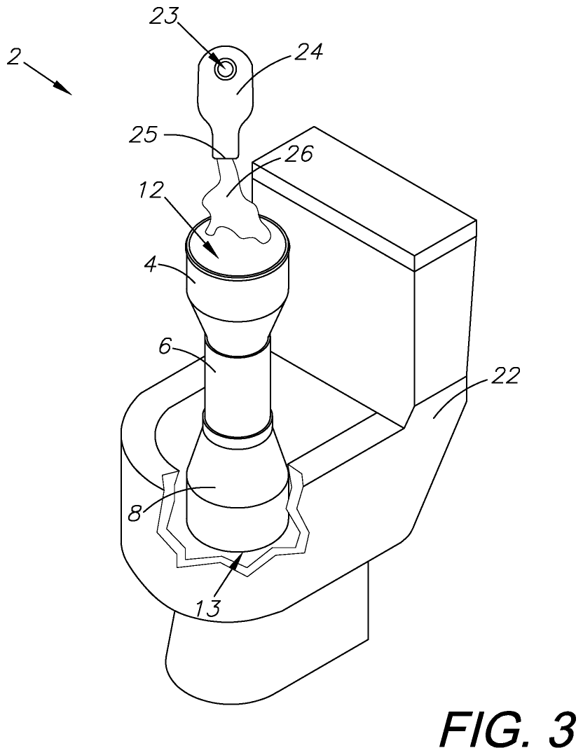

As shown in FIGS. 1-3, a preferred embodiment of the present invention would employ an ostomy disposal system 2 including a body which has a top chamber end 4 and a bottom chamber end 8 joined by a central tube 6. The top end 4 would receive effluent 26 contained in an ostomy pouch 24 being emptied through the distal end opening 25 of the ostomy pouch and into the top receiver opening 12. A lip 10 may surround the top chamber receiver opening 12 to stabilize the ostomy pouch 24 and enable the ostomate's use of both hands for the emptying process. The central tube 6 would convey the effluent 26 down into and through the disposal opening 13 of bottom end 8, which can be placed in a receptacle for sanitary disposal, such as in the bowl of a toilet 22 as shown in FIG. 3. FIG. 2 shows how the top end 4 can have threads 14 be threadedly received by a second pair of threads 16 within the top end of the central tube 6. Similarly, a third 18 and fourth 20 set of threads can join the central tube 6 with the bottom end 8. Other attachment types can be used to allow for permanent or selectively assembled orientations of the device to adjust for an ostomate's height, or the need to be seated during the emptying process (for example, by an individual in a wheelchair).

An open-end ostomy appliance consists of an adhesive wafer with a stoma opening 23 that surrounds the ostomate's stoma to allow the effluent 26 to flow directly into the pouch 24 through the stoma opening 23. The wafer adheres to the user's abdomen. The pouch may be detachable from the wafer or affixed to the wafer. The effluent 26 inside the pouch 24 is emptied via the distal end disposal opening 25 of the ostomy pouch 24. An ostomate's stoma activity may require this emptying process frequently throughout the day. In order to independently empty an ostomy pouch directly into a toilet, an ostomate must be physically able to assume one of the four commonly-used positions: seated facing forward on the toilet to empty the effluent between parted legs; seated facing the toilet tank to empty between parted legs; standing in front of the toilet bowl to empty from a distance above the toilet bowl; or kneeling in front of the toilet bowl to empty the effluent. Each of these four positions require advance preparation to line the toilet bowl with toilet paper to prevent effluent “backsplash” and reduce the resulting unsanitary effluent residue. Individuals who are physically impaired cannot manage any of the four positions and are typically dependent on caregivers to perform the task or utilize disposable ostomy pouches and accrue greater expenses for costly supplies. Additionally, at this time there are few public restroom facilities that accommodate the unique needs of ostomates; this can limit an ostomate's ability to travel or comfortably attend public functions.

FIGS. 1-3 top and bottom ends are preferably formed from cone-shaped funnels. This embodiment makes emptying the effluent 26 into the top 4 receiver opening 12 easier, and also forms a more stable base for conveying the effluent 26 into the toilet bowl 22 or other disposal receptacle via the bottom end 8 and disposal opening 13.

### III. Wall-Mounted Ostomy Disposal System 102

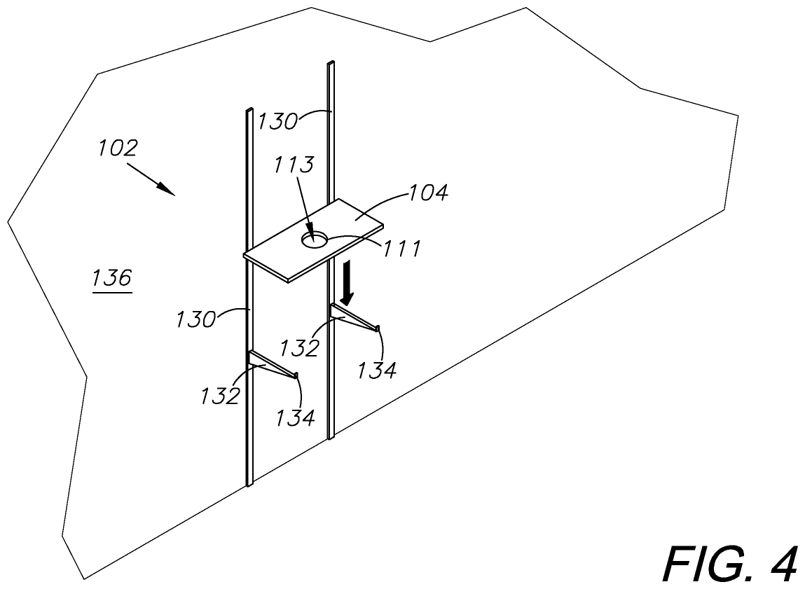

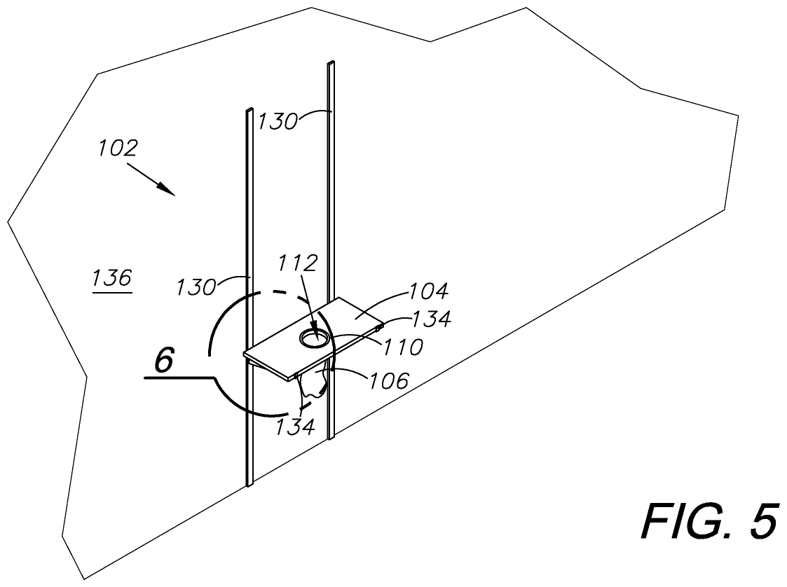

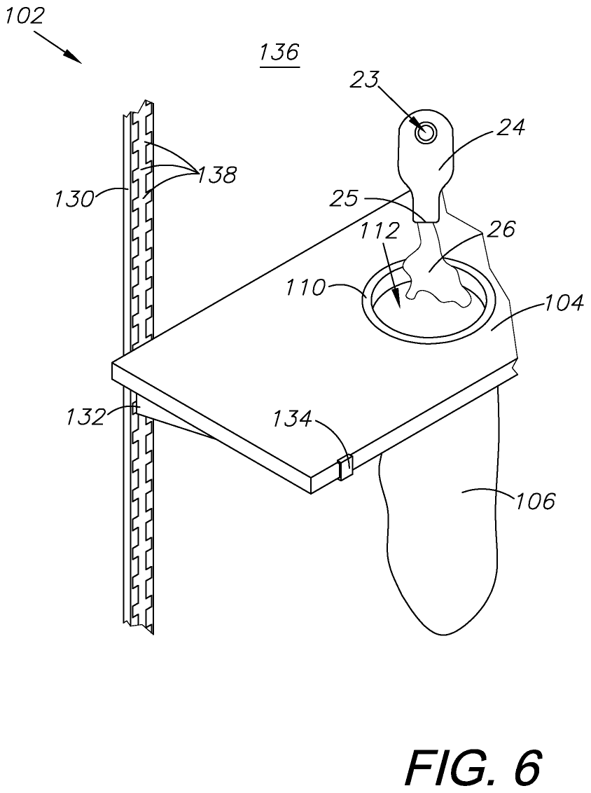

FIGS. 4-6 show an alternative embodiment, a wall-mounted ostomy disposal system 102 where a pair of mounting tracks 130 can be affixed to a wall 136. A pair of selectively adjustable brackets 132 can support a board 104 for receiving a disposable emesis bag 106 within a hole 111 having an opening 113. The emesis bag 106 includes an integrated top rim 110 which can be positioned within the top receiver opening 113 in the board 104, and which has a top opening 112 for receiving effulent. FIG. 6 shows how a series of receiver notches 138 within the mounting tracks 130 could be used to receive the selectively adjustable brackets 132 to adjust for an ostomate's height, either in a standing or seated position. These could be pin holes or other shapes so that the adjustable brackets 132 can be situated at the desired height. Optional end clips 134 can flex over the front edge of the board 104 to ensure it does not move or slip. Alternately, the adjustable brackets 132 can be integrated into or permanently affixed to the board 104.

### IV. Wall-Mounted Ostomy Disposal System 152

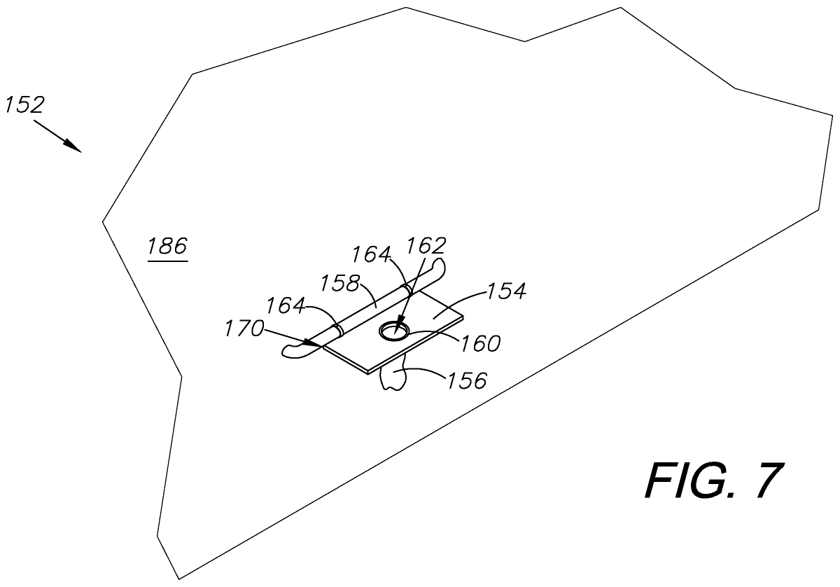

FIG. 7 shows a wall mounted ostomy disposal system 152, including a wall 186 with a grab bar 158, such as typically affixed in ADA-accessible restroom stalls or in private residences. Here, the back edge 170 of the board 154 is affixed to the grab bar 158 via straps 164, clips, or other suitable releasable means secured to the back edge of the board 154 adjacent to the wall 186. A disposable emesis bag 156 with an integrated rim 160 forming an opening 162 is inserted into the top receiver opening just as with the previous embodiment so the effluent can be emptied into the disposable emesis bag 156 by an ostomate in either a standing or seated position.

### V. Portable Ostomy Disposal System 202

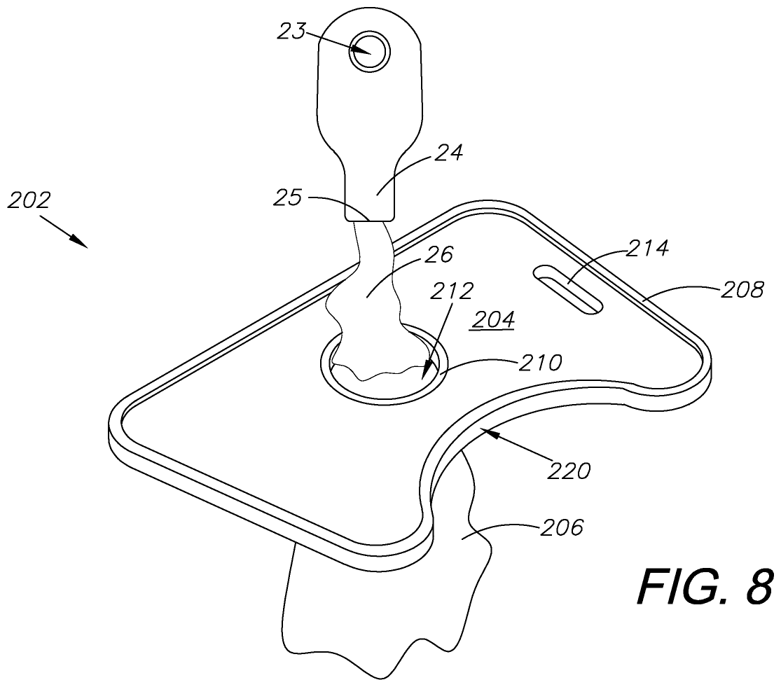

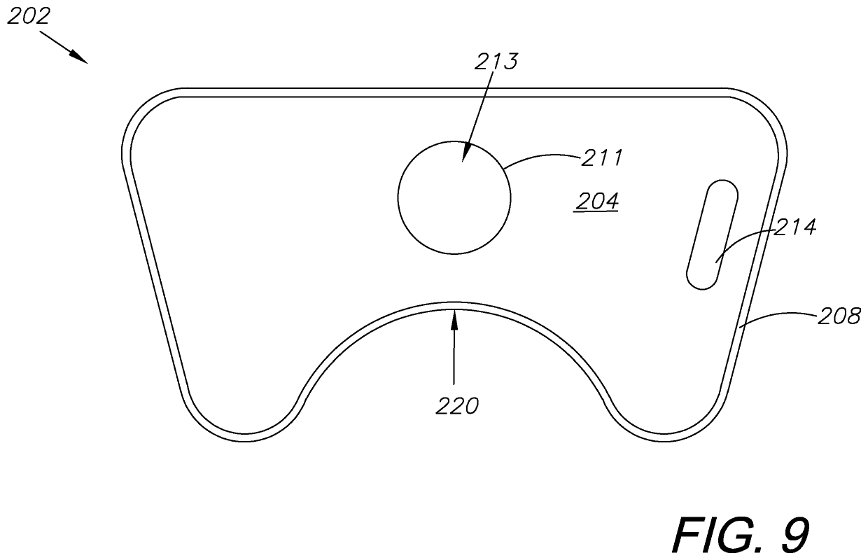

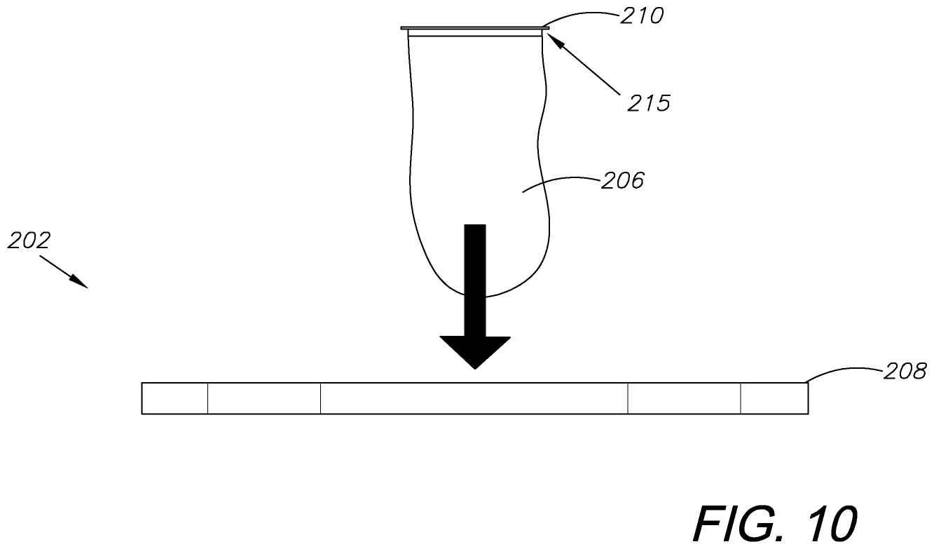

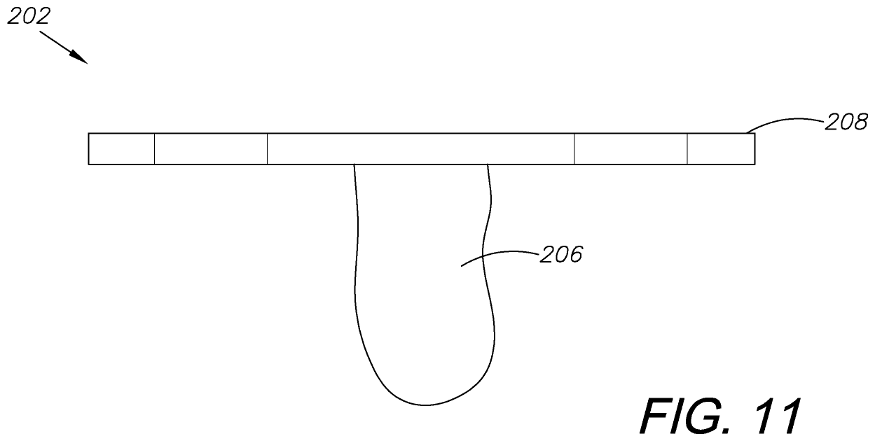

FIGS. 8-11 show a portable ostomy disposal system 202 which includes a tray board 204 with a handle 214 and a raised edge 208. The board 204 has a slip-resistant surface and raised edge 208 to aid against spilling effluent 26 when emptying an ostomy pouch 24 into a disposable emesis bag 206 with an integrated top rim 210 which is placed into a hole 211 having a receiver opening 213 in the tray 204. The integrated top rim 210 defines an opening 212 to the emesis bag 206. The tray has a curved forward face 220 designed to fit around the torso of an ostomate emptying the effluent 26 from the ostomy pouch 24 into the disposable emesis bag 206. FIGS. 10-11 show the emesis bag 206 being placed into the board 204 receiver opening and secured via the lip 215 of the integrated rim 210 of the disposable emesis bag 206.

### VI. Portable Ostomy Disposal System 302

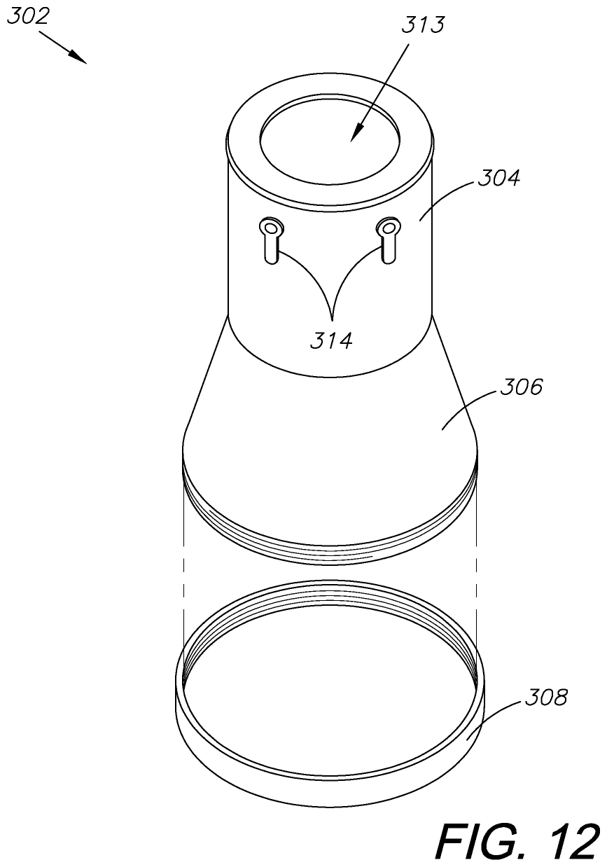

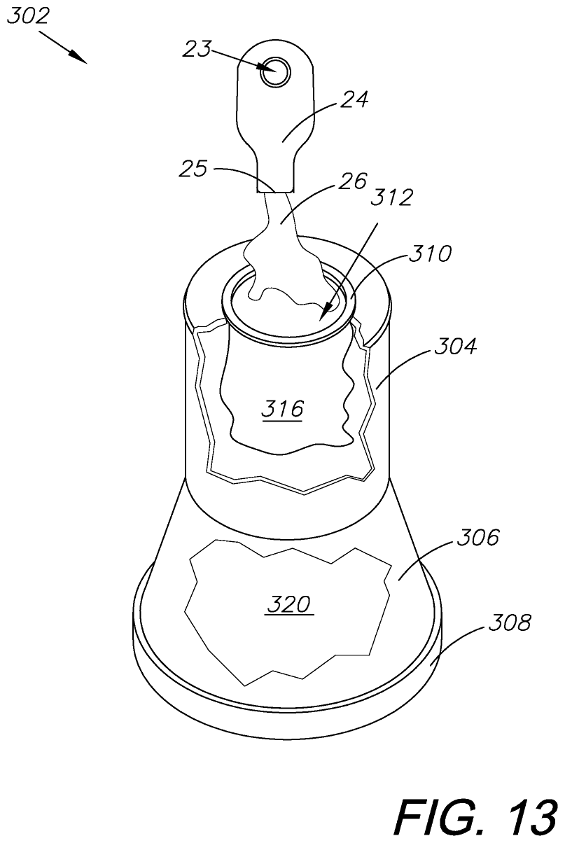

FIGS. 12-13 show a portable ostomy disposal system 302 for utilizing a disposable emesis bag 316 with an integrated rim 310. The system has a top chamber 304 with a receiver opening 313 to support the integrated rim 310 of a disposable emesis bag 316 and a bottom chamber 306 with a cap 308 which may be screwed on or otherwise connected to the bottom chamber to seal it. The upper chamber 304 can receive the effluent 26 contained within the ostomy pouch 24 as it is emptied through the distal end opening 25 into the disposable emesis bag 316 with an integrated rim 310 which is situated in the receiver opening 313. The integrated rim 310 of the disposable emesis bag 316 secures the disposal receptacle in place. The bottom chamber 306 has an interior cavity 320 which can hold reserve bags, wipes or other associated items. Optional eye hooks 314 or other appropriate fasteners may be affixed to the top chamber of the portable ostomy disposal system to accommodate a neck strap.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. An ostomy pouch emptying system comprising:
an ostomy emptying device for receiving effluent from an ostomy pouch;
said ostomy emptying device comprising a receiver opening, said receiver opening configured to receive the effluent from said ostomy pouch; and
a receptacle for receiving said effluent from said ostomy pouch in proximity with said ostomy emptying device.

2. The ostomy pouch emptying system of claim 1 comprising:
said ostomy emptying device comprising a top portion, a central tube, and a bottom portion;
said top portion having a proximal end and a distal end;
said central tube having a proximal end and a distal end;
said bottom portion having a proximal end and a distal end;
wherein said receiver opening is within said proximal end of said top portion;
wherein said distal end of said top portion is connected to said proximal end of said central tube;
wherein said distal end of said central tube is connected to a proximal end of said bottom portion; and
wherein said bottom portion proximal end with disposal opening is engaged with said receptacle.

3. The ostomy pouch emptying system of claim 2, wherein said receptacle comprises a toilet bowl.

4. The ostomy pouch emptying system of claim 2, further comprising:
said top portion comprising a tapered cone portion whereby said proximal end of said top portion is larger in diameter than said distal end of said top portion; and
said bottom portion comprising a tapered cone portion whereby said distal end of said bottom portion is larger in diameter than said proximal end of said bottom portion.

5. The ostomy pouch emptying system of claim 1, further comprising:
said ostomy emptying device comprising a board, wherein said receiver opening comprises a hole centrally located within the board;
said receptacle comprises an emesis bag having an integrated rim about a top end thereof and bag portion suspended beneath said integrated rim; and
wherein said integrated rim is configured to fit about said hole such that said bag portion is held below said board to receive said effluent.

6. The ostomy pouch emptying system of claim 5, further comprising:
a pair of mounting racks comprising a plurality of receiver notches configured to receive one of a pair of selectively adjustable brackets; and
wherein said board is configured to be placed atop said pair of selectively adjustable brackets.

7. The ostomy pouch emptying system of claim 5, further comprising at least one strap configured to secure said board to an ADA-accessible grab bar.

8. The ostomy pouch emptying system of claim 5, further comprising:
said board comprising a front end forming a curve configured to fit about an abdomen;
said board further comprising a slip-resistant surface and edge lip; and
said board further comprising a handle.

9. The ostomy pouch emptying system of claim 1, further comprising:
a top chamber comprising said receiver opening;
a bottom chamber separated from said top chamber via a barrier;
wherein said receptacle comprises an emesis bag having an integrated rim about a top end thereof and bag portion suspended beneath said integrated rim;
said integrated rim configured to fit about said receiver opening such that said bag portion is held below said board to receive said effluent; and
said bottom chamber configured to store accessories for use with said ostomy pouch emptying system.

10. The ostomy pouch emptying system of claim 9, further comprising a lid configured to be selectively removed from said bottom chamber to provide access to said bottom chamber.

11. A method of emptying an ostomy pouch, the method comprising the steps:
providing an ostomy emptying device comprising a receiver opening;
emptying effluence from an ostomy pouch into said receiver opening; and
receiving said effluence within a receptacle beneath said receiver opening.

12. The method of claim 11, further comprising the steps:
providing a top portion, a central tube, and a bottom portion of said ostomy emptying device;
connecting a distal end of said top portion to said a proximal end of said central tube; and
connecting a distal end of said central tube to a proximal end of said bottom portion.

13. The method of claim 12, wherein said receptacle comprises a toilet bowl.

14. The method of claim 12, further comprising:
wherein said top portion comprising a tapered cone portion whereby said proximal end of said top portion is larger in diameter than said distal end of said top portion; and
wherein said bottom portion comprising a tapered cone portion whereby said distal end of said bottom portion is larger in diameter than said proximal end of said bottom portion.

15. The method of claim 11, wherein:
said ostomy emptying device comprising a board;
said receiver opening comprises a hole centrally located within the board;
said receptacle comprises an emesis bag having an integrated rim about a top end thereof and bag portion suspended beneath said integrated rim; and
fitting said integrated rim about said hole such that said bag portion is held below said board to receive said effluent.

16. The method of claim 15, further comprising the steps:
providing a pair of mounting tracks comprising a plurality of receiver notches configured to receive one of a pair of selectively adjustable brackets; and
placing said board atop said pair of selectively adjustable brackets at a selectable height.

17. The method of claim 15 further comprising the step:
providing at least one strap configured to secure said board to an ADA-accessible grab bar.

18. The method of claim 15, wherein:
said board comprising a front end forming a curve configured to fit about an abdomen;
said board further comprising a slip-resistant surface and edge lip; and
said board further comprising a handle.

19. The method of claim 11, further comprising the steps:
providing a top chamber comprising said receiver opening;
providing a bottom chamber separated from said top chamber via a barrier;
wherein said receptacle comprises an emesis bag having an integrated rim about a top end thereof and bag portion suspended beneath said integrated rim;
said integrated rim configured to fit about said receiver opening such that said bag portion is held below said board to receive said effluent; and
storing accessories for use with said ostomy pouch emptying system.

20. An ostomy pouch emptying system comprising:
an ostomy emptying device comprising a top portion, a central tube, and a bottom portion;
said top portion having a proximal end and a distal end;
said central tube having a proximal end and a distal end;
said bottom portion having a proximal end and a distal end;
wherein a receiver opening is within said proximal end of said top portion;
wherein said distal end of said top portion is removably connected to said proximal end of said central tube;
wherein said distal end of said central tube is removably connected to a proximal end of said bottom portion;
wherein said bottom portion proximal end with disposal opening is engaged with said receptacle;
said top portion comprising a tapered cone portion whereby said proximal end of said top portion is larger in diameter than said distal end of said top portion; and
said bottom portion comprising a tapered cone portion whereby said distal end of said bottom portion is larger in diameter than said proximal end of said bottom portion.
